(19) United States
(12) Patent Application Publication
(10) Pub. No.: US 2024/0335655 A1
(43) Pub. Date: Oct. 10, 2024

(54) Sensing of Tissue Signals in a Deep Brain Stimulation System Using a Head-Positioned Sensing Reference Electrode

(71) Applicant: Boston Scientific Neuromodulation Corporation, Valencia, CA (US)

(72) Inventors: Rosana Esteller, Santa Clarita, CA (US); Goran Marnfeldt, Valencia, CA (US); Peter Yoo, Burbank, CA (US)

(21) Appl. No.: 18/611,384

(22) Filed: Mar. 20, 2024

Related U.S. Application Data
(60) Provisional application No. 63/494,708, filed on Apr. 6, 2023.

(51) Int. Cl. A61N 1/05; A61N 1/02; A61N 1/36; A61N 1/375

(52) U.S. Cl. CPC A61N 1/0534; A61N 1/025; A61N 1/36125; A61N 1/36139; A61N 1/3756

(57) ABSTRACT

Disclosed are solutions for providing a sensing reference electrode useful for sensing tissue signal in a Deep Brain Stimulation application. The sensing reference electrode is head-positioned and placed proximate to the patient's brain tissue, but not within the brain tissue itself where the primary sensing electrode is located. The sensing reference electrode may be placed under the scalp, within a burr hole plug used to secure the DBS leads, or within the cerebrospinal fluid just under the skull. The sensing reference electrode can comprise one or more electrodes on a scalp-implantable lead separate from the brain-implantable lead that includes the primary sensing electrodes. The sensing reference electrode can also comprise an additional electrode added to an otherwise standard brain-implantable lead, with the sensing reference electrode being significantly proximal on the lead such that it is not brain-implantable.

### CROSS REFERENCE TO RELATED APPLICATIONS

This is a non-provisional of U.S. Provisional Patent Application Ser. No. 63/494,708, filed Apr. 6, 2023, to which priority is claimed, and which is incorporated by reference in its entirety.

### FIELD OF THE INVENTION

This application relates to Implantable Medical Devices (IMDs), and more specifically to use of a head-positioned sensing reference electrode useable to sense tissue signals in a Deep Brain Stimulation System.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators (SCSs) to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) system. However, the present invention may find applicability with any stimulator device system.

A stimulator system typically includes an Implantable Pulse Generator (IPG) 10 as shown in FIG. 1A. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10, which header can comprise an epoxy for example. Although not shown, the leads can also connect to the IPG 10 via intervening lead extension (and as used herein, “leads” may refer to traditional leads as well as their extensions). Once inserted, the proximal contacts 21 connect to header contacts 24 within the lead connectors 22, which are in turn coupled by feedthrough pins 25 through a case feedthrough 26 to stimulation circuitry 28 within the case 12.

In the illustrated IPG 10, there are sixteen electrodes (E1-E16), split between two percutaneous leads 15, or contained on a single paddle lead 19. The header 23 may include two eight-electrode lead connectors 22 to support these leads. However, the type and number of leads, the number of electrodes, and the number of lead connectors in an IPG, are application specific and therefore can vary. The conductive case 12, or some conductive portion of the case, can also comprise an electrode (Ec) associated with the case.

In a DBS application, and as shown in FIG. 1B, the electrode leads (which again may utilize lead extensions) are implanted in the brain 32 (typically in the left and right sides) through holes in the skull, with the proximal ends of the leads (or extensions) tunneled through the patient's scalp and skin to the IPG 10, whose case 12 is typically implanted in the patient's torso, under the clavicle (collarbone) for example. (The IPG 10 could also be implanted in other upper areas of the patient below the neck, such as in the arm, and all such upper are positions are referred to herein as the “torso” for convenience). DBS therapy as provided by IPG 10 can alleviate Parkinsonian symptoms such as tremor and rigidity.

Referring again to FIG. 1A, IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases 30i, as shown in the example of FIGS. 2A and 2B. In the example shown, such stimulation is monopolar, meaning that a current is provided between a pole on the lead (e.g., at electrode E1) and the case electrode Ec 12. Use of monopolar stimulation is common in a DBS application. Stimulation could however also be bipolar or multipolar, in which a current is provided between two or more poles on the lead (e.g., at least one anode and one cathode). A pole on the lead can be formed by providing a current at more than one electrode as is known. For example, providing the same cathodic current to electrodes E1 and E2 would comprise a cathode pole formed effectively between the two physical positions of these electrodes, in what is known as a virtual pole. Stimulation parameters typically include amplitude (current I, although a voltage amplitude V can also be used); frequency (F); pulse width (PW) of the pulses or of its individual phases; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude+I to the tissue. Note that at any time the total current sunk from the tissue (e.g., −I at E1 during phase 30a) equals the total current sourced to the tissue (e.g., +I at Ec during phase 30a). The polarity of the currents at these electrodes can be changed: for example, during first phase 30a, Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40; and one or more current sinks 42ᵢ. The sources and sinks 40; and 42; can comprise Digital-to-Analog converters (DACs), and may be referred to as PDACs 40; and NDACs 42; in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node ei 39 is connected to an electrode Ei 16 via a DC-blocking capacitor Ci 38, for the reasons explained below. PDACs 40; and NDACs 42; can also comprise voltage sources.

Proper control of the PDACs 40; and NDACs 42; allows any of the electrodes 16 and the case electrode Ec 12 to act as anodes or cathodes to create a current (such as the pulses described earlier) through a patient's tissue, Z, hopefully with good therapeutic effect. In the example shown, and consistent with the first pulse phase 30a of FIG. 2A, electrode E1 has been selected as a cathode electrode to sink current from the tissue Z and case electrode Ec has been selected as an anode electrode to source current to the tissue Z. Thus PDAC 40c and NDAC 421 are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40; and the electrode nodes ci 39, and between the one or more NDACs 42; and the electrode nodes. Switching matrices allow one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796.

Much of the stimulation circuitry 28 of FIG. 3, including the PDACs 40; and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38, as is well known. FIG. 3 also shows that stimulation circuitry 28 can include passive recovery switches 41ᵢ, which are described further in U.S. Patent Application Publications 2018/0071527 and 2018/0140831. Passive recovery switches 41ᵢ may be closed to passively recover any charge remaining on the DC-blocking capacitors Ci 38 after issuance of the last pulse phase (e.g., second pulse phase 30b) to recover charge without actively driving a current using the DAC circuitry, as shown during duration 30c. Again, passive charge recovery is well known and not further described. Although not shown, the stimulation pulses may also be monophasic comprising single actively-driven phases (e.g., 30a), each followed by passive charge recovery (e.g., 30c).

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information and measurements, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with a compatible antenna in the IPG 10, such as a near-field magnetic-induction coil antenna 64a and/or a far-field RF antenna 64b.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller. A communication “wand” 76 coupleable to suitable ports on the computing device can include an IPG-compliant antenna such as a coil antenna 74a or an RF antenna 74b. The computing device itself may also include one or more RF antennas 74b. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with IPG communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

### SUMMARY

A method is disclosed for providing stimulation to a patient's brain using a system comprising a stimulator device, the stimulator device comprising a case and a plurality of electrodes comprising a case electrode associated with the case and other electrodes different from the case electrode, the method comprising: using stimulation circuitry within the case to provide stimulation between at least two of the plurality of electrodes; providing one or more first of the other electrodes within the patient's brain; providing one or more second of the other electrodes within the patient's head but not within the brain; and sensing a tissue signal in the brain using at least one of the first electrodes as a primary sensing electrode and using at least one of the second electrodes as a sensing reference electrode.

In one example, the case is configured for implantation within a torso of the patient. In one example, the stimulator device comprises sense amplifier circuitry within the case, wherein the tissue signal is sensed by coupling the at least one of the one or more first electrodes to a first input of the sense amplifier circuitry and by coupling the at least one of the one or more second electrodes to a second input of the sense amplifier circuitry. In one example, the one or more first electrodes are positioned on a first lead within the patient's brain, and wherein the one or more second electrodes are positioned on a second lead within the patient's head but not within the brain. In one example, the one or more second electrodes are within the patient's scalp. In one example, the case electrode comprises a conductive material of the case. In one example, the one or more first electrodes and the one or more second electrodes are positioned on a single lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, the system further comprises a burr hole plug positionable in a hole in the patient's skull. In one example, the burr hole plug comprises a plug contact. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the tissue signal comprises a neural response to the stimulation. In one example, the stimulation is provided between at least one of the first electrodes and the case electrode. In one example, the method further comprises using tissue biasing circuitry within the case to provide a common mode voltage to the patient's tissue. In one example, the common mode voltage is provided to the patient's tissue at least during the sensing of the tissue signal. In one example, the common mode voltage is provided at the case electrode. In one example, the common mode voltage is provided at the at least one of the second electrodes comprising the sensing reference electrode. In one example, the method further comprises using the sensed tissue signal to adjust the stimulation.

A stimulator device system is disclosed for providing stimulation to a patient's brain, comprising: a stimulator device comprising a case and a plurality of electrodes comprising a case electrode associated with the case and other electrodes different from the case electrode, wherein one or more first of the other electrodes are configured to be provided within the patient's brain, and wherein one or more second of the other electrodes are configured to be provided within the patient's head but not within the brain; stimulation circuitry within the case configured to provide stimulation between at least two of the plurality of electrodes; and sense amplifier circuitry within the case configured to sense a tissue signal in the brain using at least one of the first electrodes as a primary sensing electrode and using at least one of the second electrodes as a sensing reference electrode.

In one example, the case is configured for implantation within a torso of the patient. In one example, the tissue signal is sensed by coupling the at least one of the one or more first electrodes to a first input of the sense amplifier circuitry and by coupling the at least one of the one or more second electrodes to a second input of the sense amplifier circuitry. In one example, the system further comprises a first lead and a second lead, wherein the one or more first electrodes are positioned on the first lead, and wherein the one or more second electrodes are positioned on the second lead. In one example, the one or more second electrodes are configured to be positioned within the patient's scalp. In one example, the case electrode comprises a conductive material of the case. In one example, the system further comprises a lead, wherein the one or more first electrodes and the one or more second electrodes are positioned on the lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, the system further comprises a burr hole plug configured to be positioned in a hole in the patient's skull. In one example, the burr hole plug comprises a plug contact. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the tissue signal comprises a neural response to the stimulation. In one example, the stimulation is provided between at least one of the first electrodes and the case electrode. In one example, the system further comprises tissue biasing circuitry within the case configured to provide a common mode voltage to the patient's tissue. In one example, the tissue biasing circuitry is configured to provide the common mode voltage at least during the sensing of the tissue signal. In one example, the common mode voltage is provided at the case electrode. In one example, the common mode voltage is provided at the at least one of the second electrodes comprising the sensing reference electrode. In one example, the system further comprises control circuitry configured to adjust the stimulation using the sensed tissue signal.

A method is disclosed for providing stimulation to a patient's brain using a system comprising a stimulator device, the stimulator device comprising a case implantable within the patient's torso and at least one lead comprising a plurality of electrodes, the method comprising: using stimulation circuitry within the case to provide stimulation between at least two of the plurality of electrodes; providing one or more first of the electrodes within the patient's brain; providing one or more second of the electrodes within the patient's head but not within the brain; and sensing a tissue signal in the brain using at least one of the first electrodes as a primary sensing electrode and using at least one of the second electrodes as a sensing reference electrode.

In one example, the stimulator device comprises sense amplifier circuitry within the case, wherein the tissue signal is sensed by coupling the at least one of the one or more first electrodes to a first input of the sense amplifier circuitry and by coupling the at least one of the one or more second electrodes to a second input of the sense amplifier circuitry. In one example, the one or more first electrodes are positioned on a first lead within the patient's brain, and wherein the one or more second electrodes are positioned on a second lead within the patient's head but not within the brain. In one example, the one or more second electrodes are within the patient's scalp. In one example, the one or more first electrodes and the one or more second electrodes are positioned on a single lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, the system further comprises a burr hole plug positionable in a hole in the patient's skull. In one example, the burr hole plug comprises a plug contact. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the tissue signal comprises a neural response to the stimulation. In one example, the stimulation is provided between at least one of the first electrodes and a case electrode associated with the case. In one example, the method further comprises using tissue biasing circuitry within the case to provide a common mode voltage to the patient's tissue. In one example, the common mode voltage is provided to the patient's tissue at least during the sensing of the tissue signal. In one example, the common mode voltage is provided at a case electrode associated with the case. In one example, the common mode voltage is provided at the at least one of the second electrodes comprising the sensing reference electrode. In one example, the method further comprises using the sensed tissue signal to adjust the stimulation.

A stimulator device system is disclosed for providing stimulation to a patient's brain, comprising: a stimulator device comprising a case configured for implantation in a patient's torso; at least one lead comprising a plurality of electrodes, wherein one or more first of the electrodes are configured to be provided within the patient's brain, and wherein one or more second of the electrodes are configured to be provided within the patient's head but not within the brain; stimulation circuitry within the case configured to provide stimulation using at least one of the plurality of electrodes; and sense amplifier circuitry within the case configured to sense a tissue signal in the brain using at least one of the first electrodes as a primary sensing electrode and using at least one of the second electrodes as a sensing reference electrode.

In one example, the tissue signal is sensed by coupling the at least one of the one or more first electrodes to a first input of the sense amplifier circuitry and by coupling the at least one of the one or more second electrodes to a second input of the sense amplifier circuitry. In one example, the system further comprises a first lead and a second lead, wherein the one or more first electrodes are positioned on the first lead, and wherein the one or more second electrodes are positioned on the second lead. In one example, the one or more second electrodes are configured to be positioned within the patient's scalp. In one example, the system further comprises a lead, wherein the one or more first electrodes and the one or more second electrodes are positioned on the lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, the system further comprises a burr hole plug configured to be positioned in a hole in the patient's skull. In one example, the burr hole plug comprises a plug contact. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the tissue signal comprises a neural response to the stimulation. In one example, the stimulation is provided between at least one of the first electrodes and a case electrode associated with the case. In one example, the system further comprises tissue biasing circuitry within the case configured to provide a common mode voltage to the patient's tissue. In one example, the tissue biasing circuitry is configured to provide the common mode voltage at least during the sensing of the tissue signal. In one example, the common mode voltage is provided at a case electrode associated with the case. In one example, the common mode voltage is provided at the at least one of the second electrodes comprising the sensing reference electrode. In one example, the system further comprises control circuitry configured to adjust the stimulation using the sensed tissue signal.

A method is disclosed for sensing tissue signals in a patient's brain using a system comprising an implantable device, the implantable device comprising a case and a plurality of electrodes comprising a case electrode associated with the case and other electrodes different from the case electrode, the method comprising: providing one or more first of the other electrodes within the patient's brain; providing one or more second of the other electrodes within the patient's head but not within the brain; and sensing a tissue signal in the brain using at least one of the first electrodes as a primary sensing electrode and using at least one of the second electrodes as a sensing reference electrode.

In one example, the implantable device comprises sense amplifier circuitry with the case, wherein the tissue signal is sensed by coupling the at least one of the one or more first electrodes to a first input of the sense amplifier circuitry and by coupling the at least one of the one or more second electrodes to a second input of the sense amplifier circuitry. In one example, the one or more first electrodes are positioned on a first lead within the patient's brain, and wherein the one or more second electrodes are positioned on a second lead within the patient's head but not within the brain. In one example, the one or more second electrodes are within the patient's scalp. In one example, the one or more first electrodes and the one or more second electrodes are positioned on a single lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, the system further comprises a burr hole plug positionable in a hole in the patient's skull. In one example, the burr hole plug comprises a plug contact. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the case is configured for implantation within a torso of the patient. In one example, the implantable device comprises stimulation circuitry within the case, further comprising using the stimulation circuitry to provide stimulation between at least two of the plurality of electrodes. In one example, the tissue signal comprises a neural response to the stimulation. In one example, the stimulation is provided between at least one of the first electrodes and the case electrode. In one example, the implantable device comprises tissue biasing circuitry, further comprising using tissue biasing circuitry to provide a common mode voltage to the patient's tissue. In one example, the common mode voltage is provided to the patient's tissue at least during the sensing of the tissue signal. In one example, the common mode voltage is provided at the at least one of the second electrodes comprising the sensing reference electrode. In one example, the implantable device comprises stimulation circuitry to provide stimulation, and further comprising using the sensed tissue signal to adjust the stimulation.

An implantable device system is disclosed for sensing tissue signals in a patient's brain, comprising: an implantable device comprising a case and a plurality of electrodes comprising a case electrode associated with the case and other electrodes different from the case electrode, wherein one or more first of the other electrodes are configured to be provided within the patient's brain, and wherein one or more second of the other electrodes are configured to be provided within the patient's head but not within the brain; and sense amplifier circuitry within the case configured to sense a tissue signal in the brain using at least one of the first electrodes as a primary sensing electrode and using at least one of the second electrodes as a sensing reference electrode.

In one example, the tissue signal is sensed by coupling the at least one of the one or more first electrodes to a first input of the sense amplifier circuitry and by coupling the at least one of the one or more second electrodes to a second input of the sense amplifier circuitry. In one example the system further comprises a first lead and a second lead, wherein the one or more first electrodes are positioned on the first lead, and wherein the one or more second electrodes are positioned on the second lead. In one example, the one or more second electrodes are configured to be positioned within the patient's scalp. In one example, the system further comprises a lead, wherein the one or more first electrodes and the one or more second electrodes are positioned on the lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, the system further comprises a burr hole plug configured to be positioned in a hole in the patient's skull. In one example, the burr hole plug comprises a plug contact. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the case is configured for implantation within a torso of the patient. In one example, the system further comprises stimulation circuitry within the case, wherein the stimulation circuitry is configured to provide stimulation between at least two of the plurality of electrodes. In one example, the tissue signal comprises a neural response to the stimulation. In one example, the stimulation is provided between at least one of the first electrodes and the case electrode. In one example, the system further comprises tissue biasing circuitry within the case, wherein the tissue biasing circuitry is configured to provide a common mode voltage to the patient's tissue. In one example, the common mode voltage is provided to the patient's tissue at least during the sensing of the tissue signal. In one example, the common mode voltage is provided at the at least one of the second electrodes comprising the sensing reference electrode. In one example, the system further comprises control circuitry within the case configured to adjust stimulation provided by the implantable device using the sensed tissue signal.

A method is disclosed for sensing tissue signals in a patient's brain using a system comprising an implantable device, the implantable device comprising a case implantable within the patient's torso and at least one lead comprising a plurality of electrodes, the method comprising: providing one or more first of the electrodes within the patient's brain; providing one or more second of the electrodes within the patient's head but not within the brain; and sensing a tissue signal in the brain using at least one of the first electrodes as a primary sensing electrode and using at least one of the second electrodes as a sensing reference electrode.

In one example, the implantable device comprises sense amplifier circuitry with the case, wherein the tissue signal is sensed by coupling the at least one of the one or more first electrodes to a first input of the sense amplifier circuitry and by coupling the at least one of the one or more second electrodes to a second input of the sense amplifier circuitry. In one example, the one or more first electrodes are positioned on a first lead within the patient's brain, and wherein the one or more second electrodes are positioned on a second lead within the patient's head but not within the brain. In one example, the one or more second electrodes are within the patient's scalp. In one example, the one or more first electrodes and the one or more second electrodes are positioned on a single lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, the system further comprises a burr hole plug positionable in a hole in the patient's skull. In one example, the burr hole plug comprises a plug contact. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the implantable device comprises stimulation circuitry within the case, further comprising using the stimulation circuitry to provide stimulation between at least two of the plurality of electrodes. In one example, the tissue signal comprises a neural response to the stimulation. In one example, the stimulation is provided between at least one of the first electrodes and a case electrode associated with the case. In one example, the implantable device comprises tissue biasing circuitry, further comprising using tissue biasing circuitry to provide a common mode voltage to the patient's tissue. In one example, the common mode voltage is provided to the patient's tissue at least during the sensing of the tissue signal. In one example, the common mode voltage is provided at the at least one of the second electrodes comprising the sensing reference electrode. In one example, the implantable device comprises stimulation circuitry to provide stimulation, and further comprising using the sensed tissue signal to adjust the stimulation.

An implantable device system is disclosed for sensing tissue signals in a patient's brain, comprising: an implantable device comprising a case configured for implantation in a patient's torso; at least one lead comprising a plurality of electrodes, wherein one or more first of the electrodes are configured to be provided within the patient's brain, and wherein one or more second of the electrodes are configured to be provided within the patient's head but not within the brain; and sense amplifier circuitry within the case configured to sense a tissue signal in the brain using at least one of the first electrodes as a primary sensing electrode and using at least one of the second electrodes as a sensing reference electrode.

In one example, the tissue signal is sensed by coupling the at least one of the one or more first electrodes to a first input of the sense amplifier circuitry and by coupling the at least one of the one or more second electrodes to a second input of the sense amplifier circuitry. In one example, the system further comprises a first lead and a second lead, wherein the one or more first electrodes are positioned on the first lead, and wherein the one or more second electrodes are positioned on the second lead. In one example, the one or more second electrodes are configured to be positioned within the patient's scalp. In one example, the system further comprises a lead, wherein the one or more first electrodes and the one or more second electrodes are positioned on the lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, the system further comprises a burr hole plug configured to be positioned in a hole in the patient's skull. In one example, the burr hole plug comprises a plug contact. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the system further comprises stimulation circuitry within the case, wherein the stimulation circuitry is configured to provide stimulation between at least two of the plurality of electrodes. In one example, the tissue signal comprises a neural response to the stimulation. In one example, the stimulation is provided between at least one of the first electrodes and a case electrode associated with the case. In one example, the system further comprises tissue biasing circuitry within the case, wherein the tissue biasing circuitry is configured to provide a common mode voltage to the patient's tissue. In one example, the common mode voltage is provided to the patient's tissue at least during the sensing of the tissue signal. In one example, the common mode voltage is provided at the at least one of the second electrodes comprising the sensing reference electrode. In one example, the system further comprises control circuitry within the case configured to adjust stimulation provided by the implantable device using the sensed tissue signal.

A stimulator device system is disclosed for providing stimulation to a patient's brain, comprising: a stimulator device comprising a case and a plurality of electrodes, wherein one or more first of the electrodes are configured to be provided within the patient's brain; a burr hole plug configured to be positioned in a hole in the patient's skull, the burr hole plug comprising a plug contact; stimulation circuitry within the case configured to provide stimulation between at least two of the plurality of electrodes; and sense amplifier circuitry within the case configured to sense a tissue signal in the brain using at least one of the first electrodes as a primary sensing electrode and using the plug contact as a sensing reference electrode.

In one example, the case is configured for implantation within a torso of the patient. In one example, the tissue signal is sensed by coupling the at least one of the one or more first electrodes to a first input of the sense amplifier circuitry and by coupling the at least one of the one or more second electrodes to a second input of the sense amplifier circuitry. In one example, the system further comprises a first lead and a second lead, wherein the one or more first electrodes are positioned on the first lead, and wherein the one or more second electrodes are positioned on the second lead. In one example, the one or more second electrodes are configured to be positioned within the patient's scalp. In one example, the system further comprises a lead, wherein the one or more first electrodes and the one or more second electrodes are positioned on the lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the tissue signal comprises a neural response to the stimulation. In one example, the stimulation is provided between at least one of the first electrodes and a case electrode associated with the case. In one example, the system further comprises tissue biasing circuitry within the case configured to provide a common mode voltage to the patient's tissue. In one example, the tissue biasing circuitry is configured to provide the common mode voltage at least during the sensing of the tissue signal. In one example, the common mode voltage is provided at a case electrode associated with the case. In one example, the common mode voltage is provided at the at least one of the second electrodes. In one example, the system further comprises control circuitry configured to adjust the stimulation using the sensed tissue signal.

A stimulator device system is disclosed for providing stimulation to a patient's brain, comprising: a stimulator device comprising a case and a plurality of electrodes, wherein one or more first of the electrodes are configured to be provided within the patient's brain; a contact positionable within the patient's head but not within the brain, wherein the contact is configured to contact one or more second of the electrodes different from the first electrodes; stimulation circuitry within the case configured to provide stimulation between at least two of the plurality of electrodes; and sense amplifier circuitry within the case configured to sense a tissue signal in the brain using at least one of the first electrodes as a primary sensing electrode and using the contact as a sensing reference electrode.

In one example, the case is configured for implantation within a torso of the patient. In one example, the tissue signal is sensed by coupling the at least one of the one or more first electrodes to a first input of the sense amplifier circuitry and by coupling the at least one of the one or more second electrodes to a second input of the sense amplifier circuitry. In one example, the system further comprises a first lead and a second lead, wherein the one or more first electrodes are positioned on the first lead, and wherein the one or more second electrodes are positioned on the second lead. In one example, the one or more second electrodes are configured to be positioned within the patient's scalp. In one example, the system further comprises a lead, wherein the one or more first electrodes and the one or more second electrodes are positioned on the lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, one of the one or more second electrodes are configured to contact the contact when the one or more first electrodes are provided within the patient's brain. In one example, at least a portion of the contact is configured to be positioned in the patient's scalp or below the patient's skull. In one example, the tissue signal comprises a neural response to the stimulation. In one example, the stimulation is provided between at least one of the first electrodes and a case electrode associated with the case. In one example, the system further comprises tissue biasing circuitry within the case configured to provide a common mode voltage to the patient's tissue. In one example, the tissue biasing circuitry is configured to provide the common mode voltage at least during the sensing of the tissue signal. In one example, the common mode voltage is provided at a case electrode associated with the case. In one example, the common mode voltage is provided at the at least one of the second electrodes. In one example, the system further comprises control circuitry configured to adjust the stimulation using the sensed tissue signal.

An implantable device system is disclosed for sensing tissue signals in a patient's brain, comprising: an implantable device comprising a case and a plurality of electrodes, wherein one or more first of the electrodes are configured to be provided within the patient's brain; a burr hole plug configured to be positioned in a hole in the patient's skull, the burr hole plug comprising a plug contact; and sense amplifier circuitry within the case configured to sense a tissue signal in the brain using at least one of the first electrodes as a primary sensing electrode and using the plug contact as a sensing reference electrode.

In one example, the case is configured for implantation within a torso of the patient. In one example, the tissue signal is sensed by coupling the at least one of the one or more first electrodes to a first input of the sense amplifier circuitry and by coupling the at least one of the one or more second electrodes to a second input of the sense amplifier circuitry. In one example, the system further comprises a first lead and a second lead, wherein the one or more first electrodes are positioned on the first lead, and wherein the one or more second electrodes are positioned on the second lead. In one example, the one or more second electrodes are configured to be positioned within the patient's scalp. In one example, the system further comprises a lead, wherein the one or more first electrodes and the one or more second electrodes are positioned on the lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, one of the one or more second electrodes are configured to contact the plug contact when the one or more first electrodes are provided within the patient's brain. In one example, at least a portion of the plug contact is configured to be positioned in the patient's scalp or below the patient's skull. In one example, the system further comprises stimulation circuitry within the case configured to provide stimulation between at least two of the plurality of electrodes, wherein the tissue signal comprises a neural response to the stimulation. In one example, the stimulation is provided between at least one of the first electrodes and a case electrode associated with the case. In one example, the system further comprising tissue biasing circuitry within the case configured to provide a common mode voltage to the patient's tissue. In one example, the tissue biasing circuitry is configured to provide the common mode voltage at least during the sensing of the tissue signal. In one example, the common mode voltage is provided at a case electrode associated with the case. In one example, the common mode voltage is provided at the at least one of the second electrodes. In one example, the system further comprises stimulation circuitry within the case configured to provide stimulation between at least two of the plurality of electrodes, and further comprises control circuitry configured to adjust the stimulation using the sensed tissue signal.

An implantable device system is disclosed for sensing tissue signals in a patient's brain, comprising: an implantable medical device comprising a case and a plurality of electrodes, wherein one or more first of the electrodes are configured to be provided within the patient's brain; a contact positionable within the patient's head but not within the brain, wherein the contact is configured to contact one or more second of the electrodes different from the first electrodes; and sense amplifier circuitry within the case configured to sense a tissue signal in the brain using at least one of the first electrodes as a primary sensing electrode and using the contact as a sensing reference electrode.

In one example, the case is configured for implantation within a torso of the patient. In one example, the tissue signal is sensed by coupling the at least one of the one or more first electrodes to a first input of the sense amplifier circuitry and by coupling the at least one of the one or more second electrodes to a second input of the sense amplifier circuitry. In one example, the system further comprises a first lead and a second lead, wherein the one or more first electrodes are positioned on the first lead, and wherein the one or more second electrodes are positioned on the second lead. In one example, the one or more second electrodes are configured to be positioned within the patient's scalp. In one example, the system further comprises a lead, wherein the one or more first electrodes and the one or more second electrodes are positioned on the lead. In one example, the one or more second electrodes are more proximate to the case than are the one or more first electrodes. In one example, there is only one second electrode. In one example, the one or more second electrodes are separated from the one or more first electrodes by a distance greater than a distance between the one or more first electrodes from each other. In one example, the one or more first electrodes and the one or more second electrodes are differently configured. In one example, the one or more second electrodes are flexible. In one example, one of the one or more second electrodes are configured to contact the contact when the one or more first electrodes are provided within the patient's brain. In one example, at least a portion of the contact is configured to be positioned in the patient's scalp or below the patient's skull. In one example, the system further comprises stimulation circuitry within the case configured to provide stimulation between at least two of the plurality of electrodes, wherein the tissue signal comprises a neural response to the stimulation. In one example, the stimulation is provided between at least one of the first electrodes and a case electrode associated with the case. In one example, the system further comprises tissue biasing circuitry within the case configured to provide a common mode voltage to the patient's tissue. In one example, the tissue biasing circuitry is configured to provide the common mode voltage at least during the sensing of the tissue signal. In one example, the common mode voltage is provided at a case electrode associated with the case. In one example, the common mode voltage is provided at the at least one of the second electrodes. In one example, the system further comprises stimulation circuitry within the case configured to provide stimulation between at least two of the plurality of electrodes, and further comprises control circuitry configured to adjust the stimulation using the sensed tissue signal.

### BRIEF DESCRIPTION OF THE DRAWINGS

FIG. 1A shows an Implantable Pulse Generator (IPG), and FIG. 1B shows the IPG as implanted in a patient in a Deep Brain Stimulation (DBS) example, in accordance with the prior art.

FIGS. 2A and 2B show an example of stimulation pulses producible by the IPG, in accordance with the prior art.

FIG. 3 shows stimulation circuitry useable in the IPG, in accordance with the prior art.

FIG. 4 shows various external devices capable of communicating with and programming stimulation in an IPG, in accordance with the prior art.

FIG. 5 shows circuitry in an IPG having tissue signal sensing capability.

FIG. 6 shows manners in which one or more sensing electrodes can be selected to sense a tissue signal in a DBS application.

FIG. 7 shows use of one or more electrodes on a scalp-implanted lead to act as a sensing reference in a DBS application.

FIG. 8 shows a lead having a dedicated head-postionable sensing reference electrode useable during the sensing of tissue signals in a DBS application.

FIGS. 9A-9C show the insertion of the lead of FIG. 8 to different depths in the brain, and shows designs for the sensing reference electrode that promote flexibility.

FIGS. 10A and 10B shows inclusion of a plug contact on a burr hole plug useable in a DBS application, and contact between the sensing reference electrode and the plug contact to establish a sensing reference at a known location in the patient's tissue.

FIGS. 11A and 11B show different modifications to the IPG to support the sensing reference electrode in manner that does not reduce the number of standard electrodes supportable on the lead of FIG. 8.

FIGS. 12A and 12B show circuitry for the modifications of FIGS. 11A and 11B.

### DETAILED DESCRIPTION

An increasingly interesting development in pulse generator systems is the addition of sensing capability to complement the stimulation that such systems provide. For example, and as explained in U.S. Patent Application Publication 2021/0236829, it can be beneficial to sense a neural response produced by neural tissue that has received stimulation from an IPG. The '829 Publication shows an example where sensing of neural responses is useful in an SCS context, and in particular discusses the sensing of Evoked Compound Action Potentials, or “ECAPs,” in a patient's spinal cord. U.S. Patent Application Publication 2022/0040486 shows an example where sensing of neural responses is useful in a DBS context, and in particular discusses the sensing of Evoked Resonant Neural Activity, or “ERNA,” in a patient's brain. The '829 Publication further discusses the sensing of stimulation artifacts caused by stimulation, as discussed further below. Still further, pulse generator systems may sense other biometric signals from a patient's tissue unrelated to any stimulation. Collectively, any of these sensed signals from the tissue comprise a tissue signal in a stimulator system.

FIG. 5 shows basic circuitry for sensing tissue signals in an IPG 100. The IPG 100 includes control circuitry 102, which may comprise a microcontroller for example, such as Part Number MSP430, manufactured by Texas Instruments, which is described in data sheets accessible on the Internet. Other types of control circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs) in the IPG 10 as described earlier, which ASIC(s) may additionally include the other circuitry shown in FIG. 5.

FIG. 5 includes the stimulation circuitry 28 described earlier (FIG. 3), including one or more DACs (PDACs and NDACs). A bus 118 provides digital control signals to the DACs to produce currents or voltages of prescribed amplitudes and with the correct timing at the electrodes selected for stimulation. The electrode current paths to the electrodes 16 include the DC-blocking capacitors 38 described earlier.

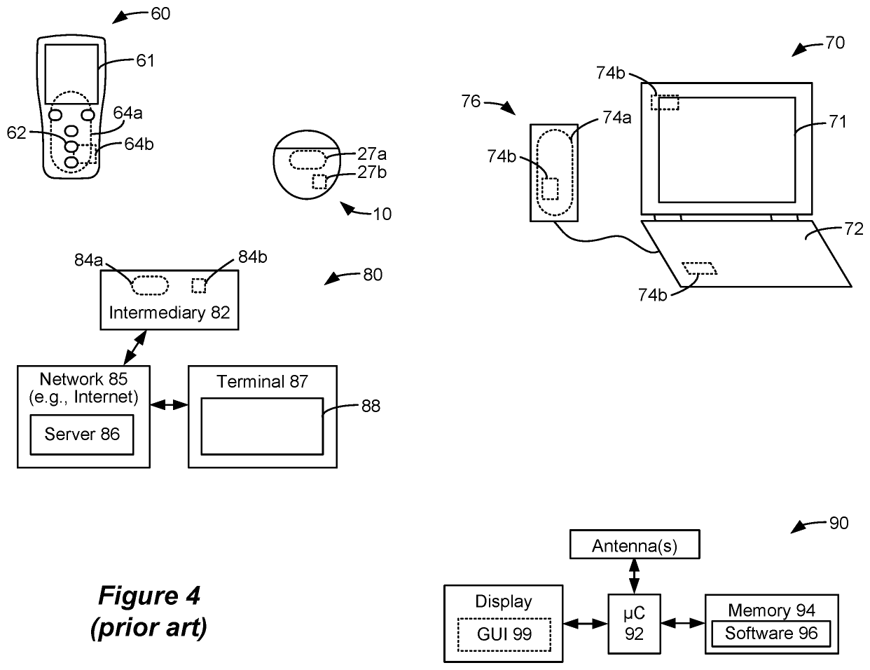

FIG. 5 also shows circuitry used to sense tissue signals. As shown, the electrode nodes 39 are input to a multiplexer (MUX) 108. The MUX 108 is controlled by a bus 114, which operates to select one or more electrode nodes, and hence to designate corresponding electrodes 16 as sensing electrodes. The sensing electrode(s) selected via bus 114 can be determined automatically by control circuitry 102 and/or a tissue signal algorithm 124, as described further below. However, the sensing electrode(s) may also be selected by the user (e.g., a clinician) via an external system 60, 70 or 80 (FIG. 4).

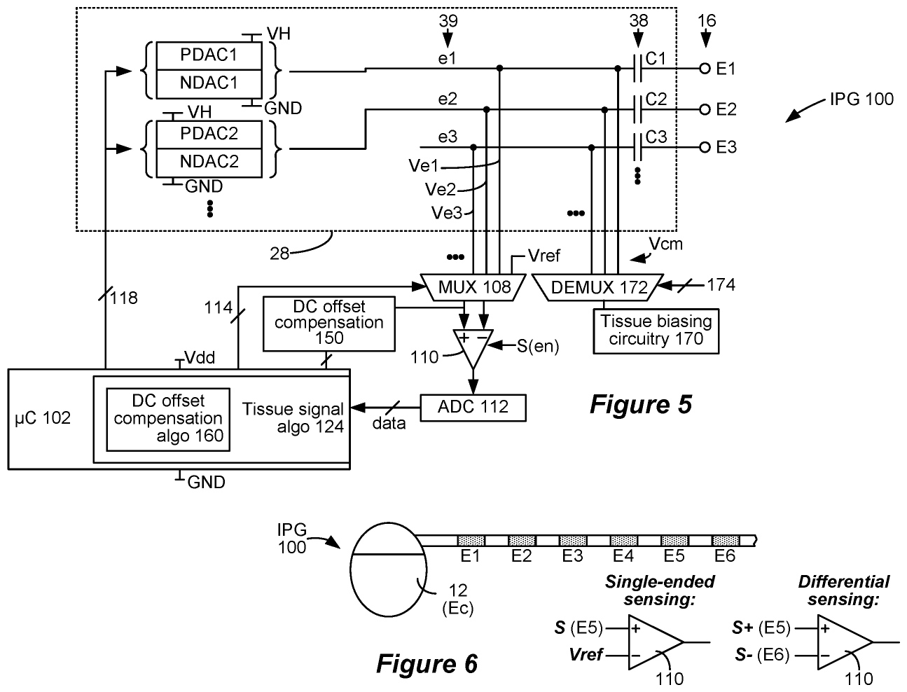

Electrode(s) selected as sensing electrodes are provided by the MUX 108 to sense amplifier (amp) circuitry 110, and sensing can occur using a single sensing electrode or differentially using two sensing electrodes. If single-ended sensing is used, a single electrode (e.g., E5) is selected as a single sensing electrode(S) and is provided to the positive input of the sense amp circuitry 110, where it is compared to a reference voltage Vref provided to the negative input, as shown in FIG. 6. The reference voltage Vref can comprise any DC voltage produced within the IPG, such as ground, the voltage of the battery 14 (Vbat), or some fraction of the compliance voltage VH (such as VH/2). If differential sensing is used, two electrodes (e.g., E5 and E6) are selected as sensing electrodes (S+ and S−) by the MUX 108, with one electrode (e.g., E5) provided to the positive input of the sense amp circuitry 110, and the other (e.g., E6) provided to the negative input. As explained in the '829 Publication, differential sensing can be useful to cancel any background voltage present in the tissue and reflected at the electrodes, as discussed further below. One skilled in the art will understand that the sense amplifier circuitry 110 comprises a differential amplifier, and thus produces an output voltage that comprises an (amplified) difference between the voltages at its positive and negative inputs. See, e.g., U.S. Patent Application Publication 2020/0305744.

Sensing of tissue signals can be further assisted by the use of techniques that equilibrate the DC voltage levels of the inputs to the sense amp. To provide such functionality, and as shown in FIG. 5, the IPG 100 can include DC offset compensation circuitry 150, and/or a DC offset compensation algorithm 160 which operates in the control circuitry 102 to control the stimulation circuitry 28 to provide such compensation. Further details concerning sense amp circuitry 110 and DC offset compensation techniques are disclosed in U.S. Patent Application Publications 2020/0305744, 2023/0173273, and 2024/0055021, which are incorporated herein by reference in their entireties.

Although only one sense amp circuit 110 is shown in FIG. 5 for simplicity, there could be more than one, such as a sense amp dedicated to each electrode node. Each sense amp could be activated as needed depending on which electrodes are selected as sensing electrodes. In another example most useful for differential sensing, some number of sense amp circuitries 110 (e.g., four) can be provided, thus allowing any eight of the electrodes to be selected to act as sensing electrodes at a given time (e.g., by one or more MUXes 108). The timing at which sensing occurs can be affected by a sensing enable signal S (en).

The analog waveform comprising the amplified tissue signal is preferably converted to digital signals by an Analog-to-Digital converter (ADC) 112, and input to the IPG's control circuitry 102. The ADC 112 can be included within the control circuitry 102's input stage as well. The control circuitry 102 can be programmed with a tissue system algorithm 124 to evaluate the tissue signal, and to take appropriate actions as a result. For example, the tissue system algorithm 124 may change the stimulation in accordance with a sensed tissue signal, and can issue new control signals via bus 118 to change operation of the stimulation circuitry 28 to affect better treatment for the patient. As explained in the above referenced '829 Publication, the tissue system algorithm 124 can extract features of the tissue signal, such as various peak heights, line widths, areas, durations, frequency components (e.g., per Fourier analysis), energies in different frequency bands, etc., which may be used to control the stimulation as just described, to determine the effectiveness of stimulation treatment, or for other reasons.

The tissue system algorithm 124 may also cause the selection of new sensing electrode(s), which can be affected by issuing new control signals on bus 114. Selecting optimal sensing electrode(s) can be important, and may be determined in light of stimulation that is being provided. In one example, sensing electrodes may be selected near enough to the electrodes providing stimulation in the electrode array 17 to allow for proper tissue signal sensing, but far enough that the stimulation doesn't substantially interfere with tissue signal sensing. See, e.g., U.S. Patent Application Publication 2020/0155019.

Tissue signals are typically small-amplitude AC signals on the order of micro Volts or milliVolts, which can make sensing difficult. The sense amp circuitry 110 needs to be capable of resolving this small signal, and this is particularly difficult when one realizes that this small signal typically rides on a background voltage otherwise present in the tissue. This background voltage may be caused by the stimulation itself—a so-called stimulation artifact—but can also result from other biometric signals unrelated to stimulation (e.g., signals resulting from heartbeats, respiration, muscular movements, etc.). As noted above, differential sensing is particularly useful because it allows the sense amp circuitry 110 to subtract any background voltages present in the tissue from the measurement, hence making tissue signals easier to resolve.

U.S. Pat. No. 11,040,202, which is incorporated herein by reference in its entirety, describes a mechanism to assist with sensing tissue signals. The '202 patent describes tissue biasing circuitry 170 (FIG. 5) that assists in tissue signal sensing by holding the tissue to a common mode voltage, Vcm, via a capacitor (such as one of the DC-blocking caps 38). This common mode voltage Vcm is preferably established at the conductive case electrode Ec, because it is large and offers low resistance to the tissue. That being said, one or more other lead-based electrodes could also be used to provide Vem as selected by demultiplexer 172 and as controlled by control signals 174. See also U.S. Patent Application Publication 2023/0138443.

As these references disclose, it is beneficial to establish the common mode voltage Vem in the tissue with reference to the power supply voltage of the DAC circuitry—i.e., the compliance voltage VH explained earlier (FIG. 3)—because the voltages in the tissue will be between this voltage and ground. Preferably, Vcm can equal approximately VH/2, although Vem can also vary somewhat from this value over time as explained in these references. When a common mode voltage Vem is provided to the tissue, AC tissue signals to be sensed will also be referenced to this voltage. This is a helpful improvement, because it tends to stabilize the DC level of the signals being input to the sense amp circuitry 110 by the sensing electrodes. That being said, generation of a particular common mode voltage Vem, while beneficial, is not required when sensing tissue signals. One skilled in the art will understand that limiting the variation of Vem is beneficial to reduce the effect of the sense amplifier circuitry 110's limited common mode rejection ratio (CMRR).

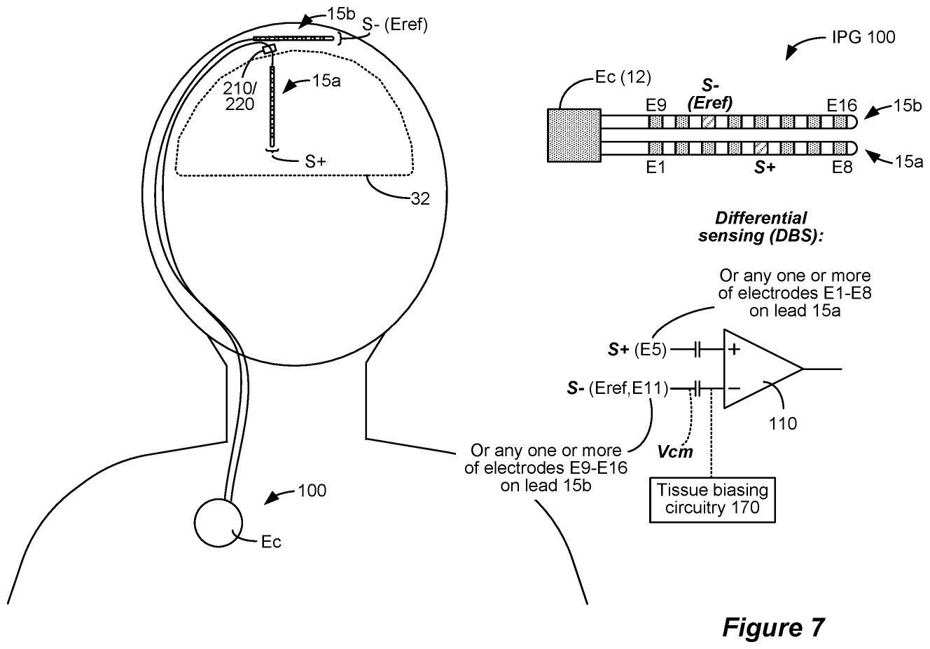

When sensing tissue signals in a DBS context, it is generally preferred to use differential sensing. As discussed above, this requires the selection of two sensing electrodes (S+ and S−) which will be input to the sense amp circuitry 110. This typically involves the selection of a suitable lead-based electrode (e.g., E5) in the brain as the primary sensing electrode (S+) and the case electrode Ec as a sensing reference electrode (S−), as shown in FIG. 7. This has benefits and drawbacks. Selection of the case electrode as a sensing reference electrode is beneficial because this torso-implanted electrode is far enough away from tissue signals being sensed that this tissue signal would not be present at the case electrode and therefore not subtracted from the measurement at the sense amp circuitry 110. By contrast, if a lead-based electrode was selected as the sensing reference electrode, and especially if that electrode (e.g., E4 or E6) is proximate to the primary sensing electrode selected (e.g., E5), the tissue signal would be significantly present at both of the selected sensing electrodes. As such, the tissue signal would be significantly subtracted out of the measurement at the sense amp circuitry 110, making it difficult or impossible to sense this signal.

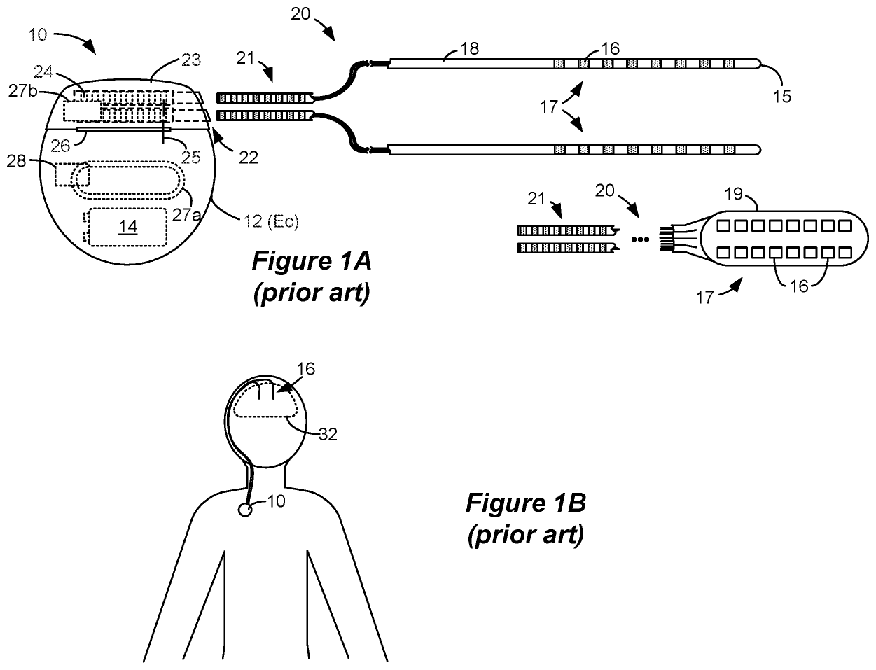

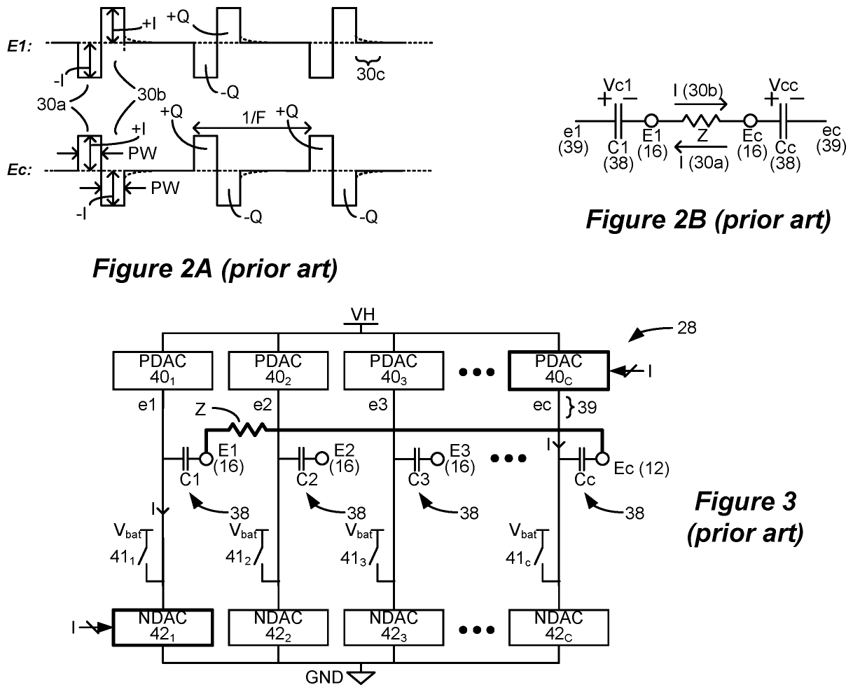

However, selection of the case electrode as a sensing reference electrode is also problematic given its location in the patient. As discussed earlier (FIG. 1B), the IPG 10 and hence the case electrode Ec is typically implanted in the patient's torso, under the clavicle, proximate to the lungs and heart. Breathing causes the chest to heave and muscles to contract and relax. Beating of the heart also causes electrical signals, such as those typically detected by an electrocardiogram. Muscle movements also cause electrical signals. These biological processes can cause electrical interference at the case electrode that can interfere with the sensing of the small-amplitude tissue signal. The position of the IPG 10 and hence the case electrode Ec will also move relative to the brain-implanted electrodes as the patient moves, which creates movement artifacts (noise) that can also affect tissue signal sensing.

The inventors address this concern by providing in a DBS application a differential sensing reference electrode (Eref, S−) that is placed proximate to the patient's brain tissue, but not within the brain tissue itself where the primary sensing electrode is located (S+). The sensing reference electrode may be placed under the scalp along the path that a DBS lead is typically tunneled. The sensing reference electrode may also be placed within a burr hole plug, i.e., within the hole drilled in the skull to accommodate the passage of the electrode lead into the brain in a DBS application. The sensing reference electrode may also be placed in the Cerebrospinal Fluid (CSF) that encases the brain with the skull. These preferred positioned for the sensing reference electrode are collected referred to as “head-positioned.” The sensing reference electrode is preferably not placed outside of the patient's head, and thus is not for example placed in the patient's neck or torso.

In these preferred positions, the sensing reference electrode is distant both from the tissue signal being sensed (e.g., by the primary sensing electrode in the brain) and from the patient's torso. As such, the sensing reference electrode provides a suitable electrode for differential tissue signal sensing that is not as susceptible electrical interference from biological processes (e.g., respiration, cardiac rhythms, etc.). The sensing reference electrode will also be positionally fixed relative to the brain-implanted primary sensing electrode, therefore allowing the differential sensing of tissue signals without risk of interference from movement artifacts.

Because the case electrode (implanted in the torso) is not selected as a sensing electrode, it can be used simultaneously to other useful ends. For example, the case electrode can be used to provide a common mode voltage (Vcm) to the tissue during sensing as explained earlier. Common mode voltage Vem can also be placed at the sensing reference electrode, as explained further below. The case electrode can be selected as one of the stimulation electrodes, e.g., during monopolar stimulation as explained earlier.

The sensing reference electrode can be configured in a number of different manners. For example, the sensing reference electrode can comprise one or more electrodes on a scalp-implantable lead separate from the brain-implantable lead that includes the primary sensing electrode. The sensing reference electrode can also comprise an additional electrode added to an otherwise standard brain-implantable lead, with the sensing reference electrode being significantly proximal such that it is not itself brain-implantable (like the remainder of the lead's electrodes).

A first example of Applicant's approach is shown in FIG. 7, and in this example the sensing reference electrode (Eref) is provided on a scalp-implantable lead 15b separate from the brain-implantable lead 15a. As is typical in a DBS application, the brain-implantable lead 15a is positioned in the patient's brain 32 through a hole 210 drilled in the patient's skull. This skull hole 210 and its accompanying burr hole plug 220 are discussed later. Such placement of the brain-implantable lead 15a allows one or more of its electrodes (e.g., E1-E8) to be selected to provide DBS stimulation therapy to the patient. As noted earlier, use of monopolar stimulation is common in a DBS application, and therefore the case electrode Ec could also be selected to provide stimulation (bipolar or multipolar stimulation could also be used). The specific electrodes selected to provide stimulation are not shown in FIG. 7. The brain-implantable lead 15a is preferably a traditional cylindrical percutaneous lead.

Scalp-implantable lead 15b may also comprise a traditional percutaneous lead, but may simply not be advanced through the skull hole 210 or its accompanying burr hole plug 220 as shown. Said differently, scalp-implantable lead 15b may not be advanced far enough to bring its distal end—i.e., any or all of its electrodes (e.g., E9-E16)-into the brain 32. As such, scalp-implantable lead 15b (or its lead extension) may be shorter than brain implantable lead 15a. Note for convenience that the scalp-implantable lead 15b is shown extending past the hole/plug 210/220 in FIG. 7 (to the right), which is also acceptable, but as just noted this lead 15b may more logically be stopped short of the hole/plug 210/220, such that its electrodes are to the left of the hole in FIG. 7.

Although not shown, scalp-implantable lead 15b may also be configured differently from the brain-implantable lead 15a. For example, scalp-implantable lead 15b may comprise a paddle lead 19, such as was described earlier (see FIG. 1A). The scalp-implantable lead 15b may be positioned at a traditional location (depth) in the patient's scalp, typical of the depth with which leads (like 15a) are tunneled in a DBS location. In one example, the lead 15b may be proximate to, or between, the epicranial aponeurosis and the loose areolar connective tissue, which one skilled will recognize as different layers within the scalp. Although not shown, because the primary purpose of scalp-implantable lead 15b is to provide a sensing reference electrode, the lead 15b may comprise a single electrode which may be larger in area than those that are used traditionally in a DBS application.

FIG. 7 explains how tissue signal sensing preferably occurs with the leads 15a and 15b positioned as shown. In the example shown, one of the electrodes (e.g., E5) on the brain-implantable lead 15a has been selected as the primary sensing electrode (S+), and one of the electrodes (e.g., E11) on the scalp-implantable lead 15b has been selected as the sensing reference electrode (S−, Eref).

Although not shown, more than one electrode on each lead 15a or 15b can be selected. For example, through proper control of the MUX 108 (FIG. 5), more than one electrode (e.g., E5 and E6) on brain-implantable lead 15a can be connected to the positive input of the sense amp circuitry 110, thus configuring these electrodes to effectively act in unison as larger primary sensing electrode (S+) to sense the tissue signal. Likewise, the MUX 108 can be controlled to connect more than one electrode (e.g., E11 and E12) on scalp-implantable lead 15b to the negative input of the sense amp circuitry 110, thus configuring these electrodes to effectively to act in unison as a larger sensing reference electrode (S−, Eref) for tissue signal sensing. In fact, all such electrodes on the scalp-implantable lead 15b (E9-E16) may be selected to act as Eref during tissue signal sensing.

Although not shown in FIG. 7, note that it is acceptable that scalp-implantable lead 15b be advanced into the brain 32 somewhat (through or past hole/plug 210/220), such that its distal-most electrodes are within the brain 32 while more-proximal electrodes are not. However, in this circumstance, it is preferred to select only these more-proximal electrodes to act as the sensing reference electrode(s), Eref, as opposed to the more-distal electrodes implanted in the brain.

Notice in FIG. 7 that the case electrode Ec is not selected to act as one of the electrodes during sensing (e.g., as the sensing reference electrode, Eref). As such, the case electrode Ec can be used for other purposes during sensing. For example, during sensing-when electrodes E5 and E11 have been selected as the sensing electrode for example—the case electrode can be used to provide a common mode voltage Vem to the tissue. As discussed earlier, this stabilizes the voltage in the tissue and assists in sensing the tissue signal. Because the case electrode Ec is large in area, and because a patient's tissue is generally quite conductive, the case electrodes serves well as a source for providing Vem to the tissue, even though it is distantly located from the electrodes on leads 15a and 15b. If the case electrode Ec is selected as a stimulation electrode (e.g., monopolar situation) during stimulation period, Vem may only be provided during sensing periods (i.e., between the stimulation periods).

Vem may also be provided to the electrode(s) selected to act as the sensing reference Eref, as shown in dotted lines. This option is beneficial because Vem can be constantly provided at Eref, even during stimulation periods when the case electrode Ec is being used for stimulation.

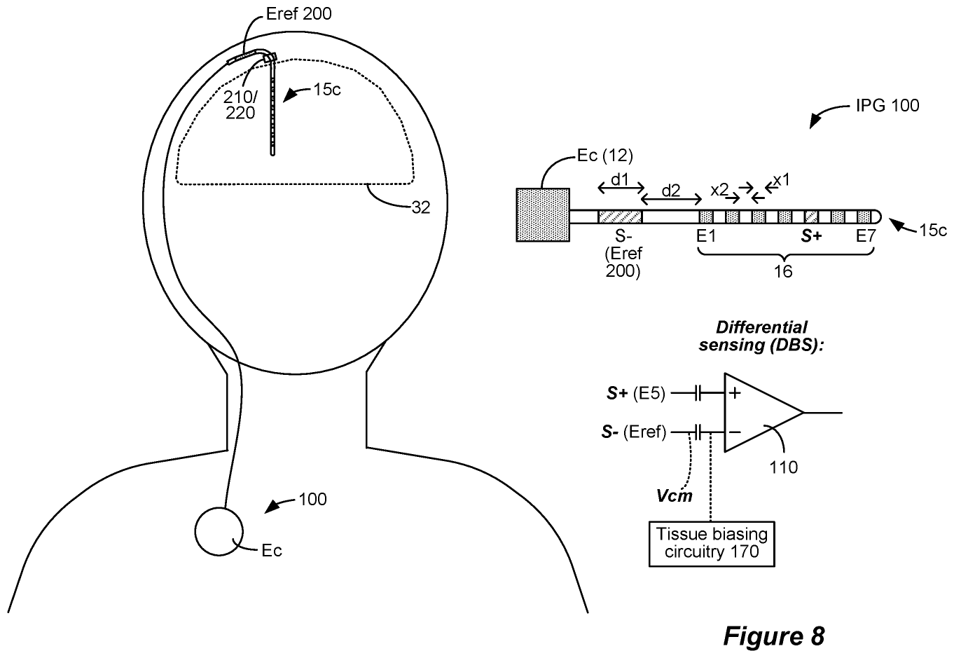

FIG. 8 shows another example for providing a sensing reference electrode (Eref) positioned at a suitable location for DBS tissue signal sensing. Unlike the example of FIG. 7 in which otherwise standard electrodes 16 on a lead 15b are selected to act as Eref, the example of FIG. 8 provides a lead 15c that is configured with a dedicated sensing reference electrode, Eref 200. Lead 15c also includes standard DBS electrodes 16 (E1-E7), which can be used to provide stimulation and to also serve as the primary sensing electrode during tissue signal sensing.

The sensing reference electrode Eref 200 is preferably configured differently from the standard electrodes 16. First, the sensing reference electrode 200 is positioned significantly proximally on the lead 15c in comparison to the distally-positioned standard electrodes 16. Thus as shown, the sensing reference electrode 200 is proximally spaced a distance d2 from the nearest standard electrode 16. Distance d2 is preferably significantly longer (e.g., 2 to 7 centimeters) than the spacing distance x2 (e.g., a few millimeters) between the standard electrodes 16 themselves. Furthermore, the sensing reference electrode 200 is preferably larger than the standard electrodes 16, for example having a length d1 (e.g., 1 to 3 centimeters) compared to the shorter length x1 of these standard electrodes (e.g., a few millimeters). One skilled will understand that the sensing reference electrode 200 is preferably larger in its area than the standard electrodes 16. Further details about the construction of sensing reference electrode 200 are discussed later. While preferred that the sensing reference electrode Eref 200 be configured differently from the standard electrodes 16, this is not strictly necessary, and instead Eref 200 can simply comprise one of, and be configured the same as, the other standard electrodes 16.

The sensing reference electrode Eref 200, especially if dedicated to acting only as a sensing reference (compare FIG. 7), may not be useable or selectable to provide stimulation. Thus, Eref may not couple to the stimulation circuitry 28 (FIG. 5), and Eref may therefore lack a dedicated PDAC/NDAC pair to drive a current at Eref. Eref may also not be useable or selectable to provide a common mode voltage Vem to the tissue, and therefore may not connect to tissue biasing circuitry 170 via DEMUX 172 for example (FIG. 5). That being said, use of the sensing reference electrode Eref 200 is not so limited, and it may also act in these capacities (to stimulate, to provide Vcm) in other examples. Thus, and although not shown, Eref may additionally be used as a return electrode during stimulation (akin to providing monopolar stimulation). Eref may provide Vcm to the tissue, in addition to acting as a sensing reference electrode, as shown again in dotted lines.

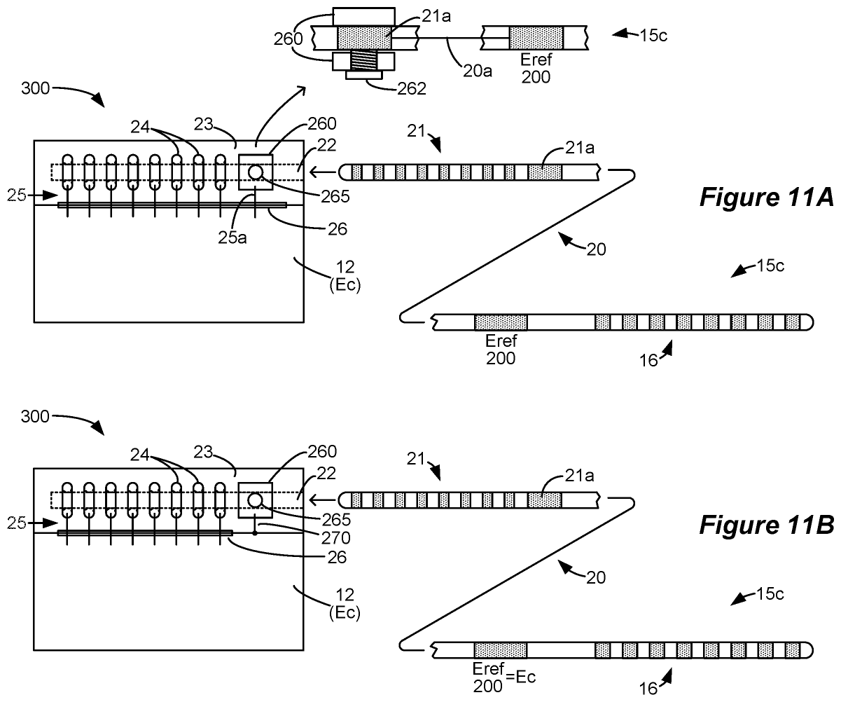

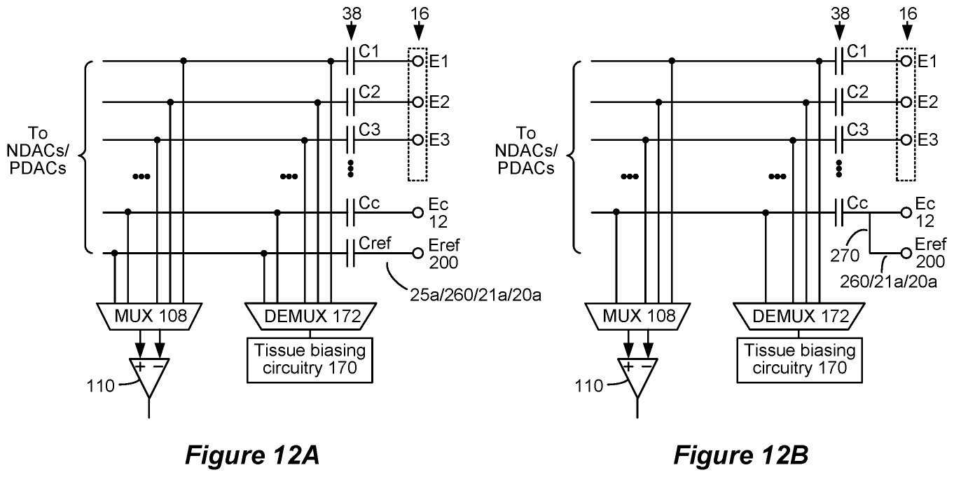

The dedicated sensing reference electrode Eref 200 on lead 15c terminates at a proximal contact 21 (FIG. 1A), which is inserted into lead connector 23 on the IPG 100 to allow Eref to be coupled to the sense amplifier circuitry 110 (e.g., via MUX 108 of FIG. 5). If it is assumed that the lead connector 23 supports eight electrodes, notice that this reduces the number of electrodes available to act as the standard electrodes 16 (e.g., E1-E7) on lead 15c. If reducing the number of standard electrodes 16 to accommodate Eref is not desirable, the number of electrodes supported by the IPG's lead connectors 21 can be increased (e.g., to nine) to support lead 15c with Eref and eight standard electrodes 16. That is, the lead connectors 21 can be modified to include nine header contacts 24. However, the IPG 100 can also be modified to supporting an additional sensing reference electrode Eref without changing the lead connectors 21 or reducing the number of electrodes they support. This is discussed later with reference to FIG. 11A-12B.

Although lead 15c is illustrated as including only a single Eref sensing reference electrode 200, the lead 15c could also include a plurality of such sensing reference electrodes, which may all act in unison to provide a sensing reference, or which may be individually selectable to act as a sensing reference.

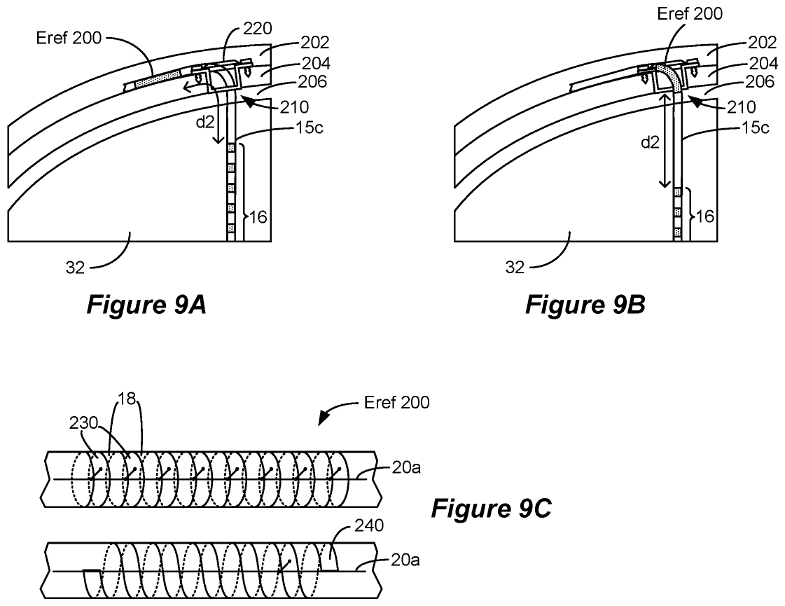

FIGS. 9A-9C show further details when lead 15c with a dedicated sensing reference electrode 200 is used in a DBS application. FIGS. 9A and 9B show the skull hole 210 that is drilled to insert the lead 15c within the brain, and the burr hole plug 220 that is eventually positioned in this hole. As one skilled in the art understands, the burr hole plug 220 serves both to cover the hole 210 once the lead(s) have been inserted, and to fixate the leads in place within the brain. The plug 220 is typically screwed to the surface of the patient's skull 204 underneath the scalp 202 as shown, and typically includes a cover to pin the fixated lead within the plug 220. Typically, the burr hole plug 220 and cover are formed of an insulative material, such as biocompatible plastic or epoxy materials.

FIGS. 9A and 9B show the lead 15c inserted to different depths in the brain 32. FIG. 9A shows a relatively shallow insertion of lead 15c. Here, the standard electrodes 16 are completely submerged in the brain 32, but the more-proximal sensing reference electrode Eref 200 is outside the brain. More specifically, and given the depth of insertion of the lead, and the distance d2 between Eref and the standard electrodes 16, Eref is located between the scalp 202 and skull 204 (e.g., between the epicranial aponeurosis and the loose areolar connective tissue as noted earlier).

FIG. 9B shows a deeper insertion of lead 15c. The standard electrodes 16 are again completely submerged in the brain 32. The sensing reference electrode Eref 200 remains outside the brain 32, but is at least partially located within the skull hole 210/plug 220. At this position, Eref 200 may also come into contact with the cerebrospinal fluid 206 between the skull 204 and brain 32. These positions for Eref are all suitable, but as noted earlier it is preferable that the Eref sensing reference electrode not be positioned within the brain itself 32 reduce the extent to which Eref 200 will receive the tissue signals being sensed. As before any one or more of the standard electrodes 16 may be selected as the primary sensing electrode (S+) to receive the tissue signal, and FIG. 8 shows use of electrode E5 for this purpose.

Because the sensing reference electrode Eref 200 may be of a larger area and longer (d1), and may be positionable where the lead 15c must bend (e.g., the hole 210/plug 220), it can be preferable to form this electrode in a flexible manner. FIG. 9C shows two examples that promote flexibility of Eref 200. In the top figure, Eref 200 is formed in electrode segments 230 as conductive rings spaced longitudinally along the lead 15c. This allows the Eref 200 electrode to bend at the locations of the flexible lead body 18 between the segments 230. One skilled will understand that these segments 230 of Eref are connected to a common lead wire 20a (FIG. 11A) inside of lead 15c to electrically short each of the segments together. In the bottom figure, Eref 200 is formed in one continuous piece of metal as a spiral 240. One skilled will understand that the material for Eref, like the standard electrodes 16, will comprise a biocompatible metal such as titanium or platinum. These are just examples of the manner in which sensing reference electrode Eref 200 can be formed on lead 15c, and other examples, including paddle-type configurations (FIG. 1A), are also possible.

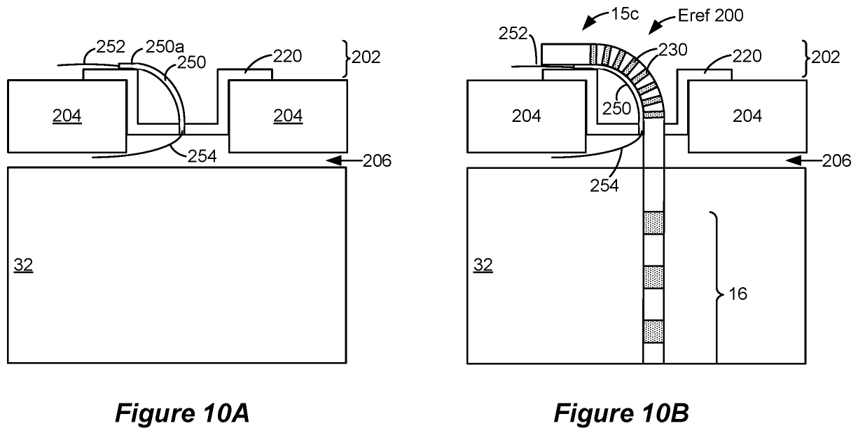

As FIGS. 9A and 9B illustrate, lead 15c may vary the location at which Eref electrode 200 is situated in the patient, especially if the lead 15c is inserted to different depths in the brain 32. This may generally be fine, but it may also be preferable that Eref 200 always be established at a set position in the patient's tissue, and FIGS. 10A and 10B illustrates manners in which this may occur. In these examples, and as best seen in FIG. 10A, the burr hole plug 220 includes a plug contact 250. This plug contact 250 may comprise a conductive biocompatible material such as titanium or platinum, and may be molded into or affixed to the insulative materials that the plug 220 is typically made from. In the example shown, the plug contact 250 is formed as an arc that proceeds from the top of the plug at a horizontal portion 250a.

The plug contact 250 is configured to electrically contact the sensing reference electrode Eref 200 when the lead 15c has been positioned in the brain 32, as best seen in FIG. 10B. Electrical contact between the plug contact 250 and Eref 200 preferably occurs regardless of the depth with which the lead 15c is inserted owing to the relative lengths of the plug contact 250 and Eref 200 (d1): these lengths will ensure that the plug contact 250 and Eref 200 will come into contact at some portions along their lengths regardless whether the lead 15c is deeply or shallowly inserted. FIG. 10B shows a relatively deep insertion, and as shown the entirety of Eref 200 (each of its segments 230) contacts the plug contact 250. In a shallower insertion, only a most-distal portion of Eref 200 (e.g., only its most-distal segments 230) would contact the plug contact 250 (e.g., at portion 250a of FIG. 10A).

Eref 200 at least partially contacting the plug contact 250 electrically shorts these contacts together, which in turn allows a sensing reference to be established at the plug contact 250, and thus allows the plug contact 250 to operate as a sensing electrode. This establishes a sensing reference at a set position in the patient's tissue, which may be preferable. If it is desired to alter this set position, the plug contact 250 may be shaped differently, and may include extensions to allow the sensing reference to also be established at nearby tissue structures. For example, FIG. 10A shows extensions 252 and 254 which may optionally be used. These extensions are electrically connected to the plug contact 250, and may be comprised of various conductive structures such as wires or meshes as appropriate for the tissue structures in which they are situated. For example, extension 252 is positionable in the patient's scalp (e.g., between the epicranial aponeurosis and the loose areolar connective tissue as mentioned earlier). Extension 254 is positionable below the skull 204, such as within the cerebrospinal fluid 206. These extensions may be considered part of the plug contact 250 itself, and the plug contact 250 and/or any extensions could be configured differently in other examples to place the sensing reference at particular locations in the patient's tissue.

While FIGS. 10A and 10B show contact 250 in conjunction with a burr hole plug 220, contact 250 may also not be associated with the burr hole plug, but may instead comprise a different implantable structure configured to electrically couple with Eref 200 on the lead 15c to provide a sensing reference at a set or desired location in the patient's tissue. Contact 250 may also comprise a portion of the lead 15c itself.

As discussed earlier, lead 15c can be used with traditional legacy-designed IPGs, although supporting Eref 200 as a sensing reference means that the number of supported distally-positioned standard electrodes 16 must decrease by one (e.g., from eight to seven). However, this is not necessarily the case, and instead the IPG can be modified to support Eref 200 as well as a traditional number (e.g., eight) of standard electrodes 16. FIGS. 11A and 11B shows different examples of such a modified IPG 300 that supports lead 15c with Eref 200 as well as eight standard electrodes 16.

In these examples, the IPG 300 relies upon a set screw block 260 in the header 23 of the IPG 300 to act as a conductor to provide a signal to the Eref sensing reference electrode 200 on the lead 15a. The header 23 of IPG 300 as described before can include one or more lead connectors 22 to receive proximal contacts 21 of lead 15c. In the example of FIGS. 11A and 11B, lead 15c has nine proximal contacts 21, one of which (21a) couples to the Eref sensing reference electrode 200 (e.g., its various segments 230 if this electrode 200 is multi-segmented) via a particular one of the lead wires 20a. The set screw block 260, as one skilled in the art will understand, comprises a structure in the header that allows the lead 15c (its proximal contacts 21) to be firmly held in place once the lead 15c has been inserted into the lead connector 22. The set screw block 260 includes an opening 265 for receiving a set screw 262, and as shown in the top-down view in FIG. 11A, this set screw 262 provides a perpendicular force to the long axis of the proximal contacts 21 to hold them firmly in place within the header and in electrical contact with the header contacts 24.

In the examples of FIGS. 11A and 11B, affixing the proximal contacts 21 into the lead connector 22 positions proximal contact 21a in contact with the set screw block 260. Because the set screw block 260 is conductive, application of the set screw 262 forces the proximal contact 21a into physical and electrical contact with the set screw block 260. This allows the proximal contact 21a, and hence Eref 200, to be electrically shorted to the set screw block 260, and in turn to different nodes in the IPG's circuitry, as explained next.

In the example of FIG. 11A, the set screw block 260 is connected to a particular one 25a of the feedthrough pins 25 which passes through the feedthrough 26 between the header 23 and the interior of the case 12. This feedthrough pin 25a can be connected to the circuitry in different fashions, and an example is shown in FIG. 12A. At a minimum, feedthrough pin 25a—and Eref 200 via connections 260, 21a, and 20a—is coupleable to the sense amp circuitry 110 (e.g., via MUX 108). This allows Eref 200 to be selected as sensing reference electrode as explained previously, and consistent with its primary function. Additionally, feedthrough pin 25a may be connected to the stimulation circuitry 28 (The PDAC/NDAC circuitry) and to the tissue biasing circuitry 170 (via DEMUX 172). The allows Eref 200 to additionally act as a stimulating electrode, or as an electrode to provide a common mode voltage (Vcm) to the tissue. As shown, the feedthrough pin 25a may be connected to the circuitry via a DC blocking capacitor 38, Cref, just as are the other electrodes, although this isn't strictly required.

In FIG. 11B, the set screw block 260 is coupled to the case, i.e., to the case electrode Ec 12, via a conductor 270 positioned within the header 23. Through connections 21a and 20a, this shorts the Eref sensing reference electrode 200 to the case electrode Ec, as shown in the circuitry diagram of FIG. 12B. This allows Eref 200 to be controlled just as the case electrode is controllable, e.g., to provide stimulation, or to provide a common mode voltage (Vcm) to the tissue. It also allows Eref 200 (and the case electrode Ec) to act as a sensing reference electrode. If Eref/Ec is selected to act as a sensing reference, there may be concerns that the case electrode will impart noise to the tissue signals being sensed. However, this concern is mitigated by the head-mounted positioning of Eref 200.

While the various embodiments of the invention have been described as useful in a DBS application, and in particular in a DBS application in which the case of the IPG is implantable within the torso of the patient, one skilled in the art will recognize that the embodiments are not so limited. Instead, the various embodiments can be used in any implantable stimulation device (e.g., SCS devices), and regardless where the case electrode is implanted in the patient. Various embodiments as disclosed can also be used in the context of external trial stimulators, in which leads are implanted in the patient, which such lead communicating transcutaneously with an external trial stimulator located external to the patient. See, e.g., U.S. Pat. No. 9,259,574 (discussing external trail stimulators).

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for providing stimulation to a patient's brain using a system comprising a stimulator device, the stimulator device comprising a case and a plurality of electrodes comprising a case electrode associated with the case and other electrodes different from the case electrode, the method comprising:
using stimulation circuitry within the case to provide stimulation between at least two of the plurality of electrodes;
providing one or more first of the other electrodes within the patient's brain;
providing one or more second of the other electrodes within the patient's head but not within the brain; and
sensing a tissue signal in the brain using at least one of the first electrodes as a primary sensing electrode and using at least one of the second electrodes as a sensing reference electrode.

2. The method of claim 1, wherein the case is configured for implantation within a torso of the patient.

3. The method of claim 1, wherein the stimulator device comprises sense amplifier circuitry within the case, wherein the tissue signal is sensed by coupling the at least one of the one or more first electrodes to a first input of the sense amplifier circuitry and by coupling the at least one of the one or more second electrodes to a second input of the sense amplifier circuitry.

4. The method of claim 1, wherein the one or more first electrodes are positioned on a first lead within the patient's brain, and wherein the one or more second electrodes are positioned on a second lead within the patient's head but not within the brain.

5. The method of claim 4, wherein the one or more second electrodes are within the patient's scalp.

6. The method of claim 1, wherein the case electrode comprises a conductive material of the case.

7. The method of claim 1, wherein the one or more first electrodes and the one or more second electrodes are positioned on a single lead.

8. The method of claim 7, wherein the one or more second electrodes are more proximate to the case than are the one or more first electrodes.

9. The method of claim 8, wherein there is only one second electrode.

10. The method of claim 8, wherein the one or more second electrodes are separated from the one or more first electrodes by a distance greater than a distance between the one or more first electrodes from each other.

11. The method of claim 7, wherein the one or more first electrodes and the one or more second electrodes are differently configured.

12. The method of claim 11, wherein the one or more second electrodes are flexible.

13. The method of claim 7, wherein the system further comprises a burr hole plug positionable in a hole in the patient's skull.

14. The method of claim 13, wherein the burr hole plug comprises a plug contact.

15. The method of claim 14, wherein one of the one or more second electrodes are configured to contact the plug contact when the one or more first electrodes are provided within the patient's brain.

16. The method of claim 15, wherein at least a portion of the plug contact is configured to be positioned in the patient's scalp or below the patient's skull.

17. The method of claim 1, wherein the tissue signal comprises a neural response to the stimulation.

18. The method of claim 1, wherein the stimulation is provided between at least one of the first electrodes and the case electrode.

19. The method of claim 1, further comprising using tissue biasing circuitry within the case to provide a common mode voltage to the patient's tissue at least during the sensing of the tissue signal.

20. A stimulator device system for providing stimulation to a patient's brain, comprising:
a stimulator device comprising a case and a plurality of electrodes comprising a case electrode associated with the case and other electrodes different from the case electrode, wherein one or more first of the other electrodes are configured to be provided within the patient's brain, and wherein one or more second of the other electrodes are configured to be provided within the patient's head but not within the brain;
stimulation circuitry within the case configured to provide stimulation between at least two of the plurality of electrodes; and
sense amplifier circuitry within the case configured to sense a tissue signal in the brain using at least one of the first electrodes as a primary sensing electrode and using at least one of the second electrodes as a sensing reference electrode.
